(19) United States
(12) Patent Application Publication
(10) Pub. No.: US 2011/0091572 A1
(43) Pub. Date: Apr. 21, 2011

(54) ACNE TREATMENT COMPOSITIONS COMPRISING NANOSILVER AND USES

(76) Inventor: Richard E. Davidson, Delray Beach, FL (US)

(21) Appl. No.: 12/996,481

(22) PCT Filed: Jun. 5, 2009
(86) PCT No.: PCT/US09/46440

§ 371 Date: Dec. 6, 2010

Related U.S. Application Data
(60) Provisional application No. 61/129,132, filed on Jun. 5, 2008.

(51) Int. Cl. A61K 33/38; A61P 17/10; A61P 17/00; A61Q 19/10; A61P 3/02; B82Y 5/00

(52) U.S. Cl. 424/618; 977/773; 977/810; 977/915

(57) ABSTRACT

This invention is directed to improved compositions for treatment of acne and related methods. In some embodiments, the present invention is directed to compositions comprising a topical vehicle, nanosilver, acne medicine and soothing agents, effective for treating acne without irritation.

### FIELD OF THE INVENTION

This invention is directed to improved compositions for treatment of acne and related methods. In some embodiments, the present invention is directed to compositions comprising a topical vehicle, nanosilver, acne medicine and soothing agents, effective for treating acne without irritation.

### BACKGROUND OF THE INVENTION

Acne vulgaris is a common, chronic disease caused by obstruction of the sebaceous glands and associated hair follicles in the skin of a patient's face and/or body, and which is characterized by the formation of pimples on the skin. Although sebum, an oily substance, is naturally made by the skin for protection and lubrication, the sebaceous glands can become blocked by dead skin cells, dirt, pollutants, and cosmetics, or become susceptible to blockage due to factors such as improper diet, stress, poor hygiene, genetics, steroidal medications, or hormonal changes.

If bacteria are trapped in blocked sebaceous glands, infections can develop, which can lead to inflammation. When bacteria on the skin are trapped in oil, a nutrient-rich anaerobic environment is established, which stimulates bacterial division. Inflammation near the skin surface results in a pustule, a small collection of pus at or near the skin surface. Deeper inflammation can form a papule, a small, solid elevation in the skin that does not contain pus. Still deeper inflammation can result in the formation of a closed sac called a cyst, or hard swellings called nodules.

Acne breakouts can be visually unappealing, itchy, and painful. Furthermore, inflamed papules can infect surrounding areas when they rupture, causing more pimples and, upon healing, scarring. Repeated acne breakouts can also cause social stigmatization, especially during puberty.

Acne often first appears during adolescence. It is estimated that 85% of adolescents are affected by acne. Smith, J A et al., Impact of Acne on Quality of Life in Adolescents, p. 113, in Care Management of Skin Diseases: Life Quality and Economic Impact (Rajagopalan, R. et al., eds), 1998. However, acne does not only affect adolescents: by some estimates, about 25% of men and 50% of women will be affected by acne at some point during their adult lives. Acne Resource Center (http://www.acne-resource.org/acne-skin-care/why-adults.html).

Considering the large number of people affected by acne and the number of treatments currently available, the continued high prevalence of this condition indicates that most, if not all, current therapies are inadequate. Thus, there remains a need in the art for improved acne treatments.

### SUMMARY OF THE INVENTION

The present invention combines the anti-bacterial properties of nanosilver, the oil-removing and other acne-treating properties of salicylic acid or other accepted acne medicine, and moisturizing and soothing ingredients to create a unique formulation that treats the underlying causes of acne (for example, bacteria and oil) and also ameliorates or reduces the harsh effects of the treatment itself.

Accordingly, one aspect of the present invention provides a composition comprising a topical vehicle, nanosilver, acne medicine, and soothing agents, effective for treatment of acne without irritation. This composition can be in solid or liquid form. In some embodiments, the composition can also include moisturizing agents. In some embodiments, it can include cleansing agents. In some embodiments, the nanosilver can be delivered in a nanosilver delivery system.

In some embodiments, the acne medicine can be, without limitation, salicylic acid, benzoyl peroxide, retinoic acid, and/or combinations thereof.

In some embodiments, the soothing agents can include, without limitation, sodium palmate, sodium palm kernelate, butyrospermum parkii (i.e., shea butter), menthe piperita (i.e., peppermint) leaf oil, sericin, pyridoxine (a form of vitamin B6), retinyl palmitate and/or other forms of vitamin A, tocopheryl acetate and/or other forms of vitamin E, lauryl laurate, hyaluronic acid, aloe barbadensis leaf juice powder, euterpe oleracea (i.e., acai berry) fruit extract, riboflavin (i.e., vitamin B2), thiamin HCl and/or other forms of vitamin B1, and/or combinations thereof.

In some embodiments, the vehicle can include, without limitation, water, glycerin, titanium dioxide, zea mays (i.e., corn) starch, hydrolyzed corn starch, hydrozlyzed starch octenylsuccinate, synthetic wax, sodium chloride, cocamidopropyl dimethylamine, hydroxyethyl behenamidopropyl dimonium chloride, pentaerythrityl tetraisostearate, glycerol stearate, tetrasodium EDTA, silica, silica dimethyl silylate, butylene glycol, sodium chondroitin sulfate, atelocollagen, butylparaben, methylparaben, phenoxyethanol, propylparaben, ethylparaben, and/or isobutylparaben.

In another aspect, the present invention provides methods of treating acne in a patient in need thereof comprising administering a therapeutically effective amount of one of the compositions of the present invention to the patient, thereby treating the acne. In some embodiments, the composition is administered topically. In some embodiments, the composition is applied to the patient's face and/or body and remains there for at least about six minutes. In some embodiments, the patient is a human of adolescent age.

### DETAILED DESCRIPTION

Embodiments of the invention are discussed in detail below. In describing embodiments, specific terminology is employed for the sake of clarity. However, the invention is not intended to be limited to the specific terminology so selected. A person skilled in the relevant art will recognize that other equivalent parts can be employed and other methods developed without parting from the spirit and scope of the invention. All references cited herein are incorporated by reference as if each had been individually incorporated.

The term “nanosilver” encompasses silver particles of nano-scale size. For example, “nanosilver” may encompass silver particles from about 5 to about 100 nanometers in diameter. However, as a person of skill in the art would appreciate, silver particles of greater or smaller size are also encompassed within the term “nanosilver” if the particles are effective when used according to the embodiments of the present invention, for example, if they have a surface-to-volume ratio that permits them to exhibit antibacterial and antimicrobial action in the disclosed compositions and methods. The terms “nanosilver,” “silver nanoparticles,” and “nanosilver particles,” as used herein, are substantially equivalent terms. “Nanoparticles,” as used herein, is generally intended to refer to silver nanoparticles.

Nanosilver particles can be coated or uncoated. For example, nanosilver particles can be incorporated into a delivery system. “Nanosilver delivery system” refers to nanosilver that is packaged or coated in a way that may facilitate or improve the activity of the nanosilver in the compositions or methods of the present invention. For example, the nanosilver may be plated onto silica microspheres, as in Bugla-Ploskonska, G. et al, Bactericidal properties of silica particles with silver islands located on the surface, International Journal of Antimicrobial Agents 29:6, 746-748 (June 2007). Without wishing to be bound to any particular theory, it is believed that the nanosilver particles are captured in pores in the silica microspheres, which then deliver the nanosilver particles to the skin. Nanosilver particles may be coated with titanium dioxide to form a nanosilver delivery system. Exemplary nanosilver delivery systems can be obtained from Benvict Chemical Co., Ltd., Taiwan; Utopia Silver Supplements, Utopia, Tex.; Active Concepts LLC, Piscataway, N.J.; Kelly Colloidal Silver, Peoria, Ariz.; and Purest Colloids, Inc., Westhampton, N.J.

“Acne medicine” encompasses the ingredient or ingredients of the compositions of the present invention that are effective in treating, reducing the symptoms or effects of, or preventing or avoiding the progression of acne. For example, acne medicines approved for use in products labeled as acne medications include salicylic acid, benzoyl peroxide and retinoic acid. As used herein, “acne medicine” and acne medication” are substantially equivalent terms.

“Acne” refers to the condition and related symptoms as it is commonly understood in the art. For example, “acne” encompasses acne vulgaris, a common, chronic disease caused by obstruction of the sebaceous glands and associated hair follicles in the skin of a patient's face and/or body, and which is characterized by the formation of pimples on the skin. “Acne” also encompasses symptoms commonly associated with acne, such as oily skin, pimples including blackheads or whiteheads, pustules, papules, nodules, cysts and/or related scarring.

“Topical,” when used in the context of medications, refers to application to the outer skin surface. “Topical vehicle” refers to any pharmaceutical carrier or delivery system that, when combined with one or more active agents, is appropriate for application to the outer surface of the skin.

“Therapeutically effective,” when used in conjunction with delivery of an active agent, means sufficient to bring about a satisfactory result or with respect to at least one condition or symptom thereof that the user intends to address by application of the composition comprising an active agent. As used herein, “active agent” means a compound or composition that can alleviate one or more symptoms or effects of an illness or condition. The therapeutically effective dose for each user will depend upon a variety of factors: the type and degree of the response to be achieved; activity of the specific agent or composition employed; the specific agents or composition employed; the age, body weight, general health, gender and diet of the patient and/or user; the time of administration; the duration of the treatment; drugs used in combination or coincidental with the specific agent; and like factors well known in the art. For example, it is well within the skill of the art to start doses of the agents at levels lower than those required to achieve the desired therapeutic effect and to gradually increase the dosages until the desired effect is achieved. Treatment may be for an acute or a chronic condition, and the therapeutically effective amount may be a single dose or multiple doses over a period of a day, days, weeks, months, or longer, as would be apparent to those skilled in the art.

The terms “treat” and “treatment” refer to both therapeutic treatment and prophylactic or preventative measures, wherein the object is to prevent, slow the progression of, or lessen an undesired physiological condition, disorder or disease, or obtain beneficial or desired results. For purposes of this invention, beneficial or desired results include, but are not limited to, alleviation of symptoms; diminishment of the extent of the condition, disorder or disease; stabilization (i.e., not worsening) of the state of the condition, disorder or disease; delay in onset or slowing of the condition, disorder or disease progression; amelioration of the condition, disorder or disease state; remission, whether partial or total, whether detectable or undetectable; or enhancement or improvement of the condition, disorder or disease. “Treatment” includes eliciting a significant response, without excessive levels of side effects.

“Irritation” refers undesirable effects to the skin surface area to be treated. Irritation can include, for example, drying, inflammation, reddening, flaking, scarring, or the results of infection. “Without irritation” refers, for example, to the avoidance or mitigation of these effects after application of the compositions of the present invention to a patient's skin.

“Salicylic acid” refers to both natural and synthetic forms of salicylic acid, as well as to derivatives and variants, for example salts or metabolites thereof, that can bring about substantially the same effects as salicylic acid when used in an acne medicine. “Salicylic acid” can also refer to natural sources of salicylic acid. For example, “salicylic acid” encompasses willow bark extracts, also known as Salix alba bark extract or Salix nigra bark extract, which are natural sources of salicylic acid.

Benzoyl peroxide, also known as dibenzoyldioxidane, is a common active agent in acne medicines. As used herein, “benzoyl peroxide” can also refer to derivatives, for example salts or metabolites thereof, that can bring about substantially the same effects as benzoyl peroxide when used as an active ingredient in an acne medication.

“Retinoic acid” refers to one or more of a class compounds related to vitamin A. “Retinoic acid” and “retinoid,” as used herein, have substantially the same meanings. “Retinoic acid” encompasses, for example, 13-cis retinoic acid, also referred to as isotretinoin, which is effective in treating acne. However, as would be appreciated by a person of skill in the art, any derivative of vitamin A that is effective in treating acne is encompassed within the definition of “retinoic acid.”

“Polyquaternium-67” refers to a polycationic polysaccharide polymer. Polyquaternium compounds comprise hydrophobically-modified quaternized ammonium groups grafted to a cellulosic polymer chain. Polyquaternium-67 is one such polymer. However, as used herein, “polyquaternium-67” can be understood to refer to any polyquaternium compound that can be employed in the compositions of the present invention. In some embodiments, Polyquaternium-67 may be used as a coating for hyaluronic acid and salicylic acid, in order to deliver these ingredients more effectively to the skin.

“Soothing agent” encompasses any ingredient that can prevent, reduce, attenuate or alleviate the irritation that can result from application of some acne medications, such as those of the present invention. “Soothing agent” includes, for example, soothing plant extracts. Examples of such soothing agents include, without limitation, sodium palmate, sodium palm kernelate, butyrospermum parkii (shea butter), menthe piperita (peppermint) leaf oil, sericin, pyridoxine (vitamin B6), retinyl palmitate (vitamin A), tocopheryl acetate (vitamin E), lauryl laurate, hyaluronic acid, aloe barbadensis leaf juice powder, euterpe oleracea (acai berry) fruit extract, riboflavin (vitamin B2), and thiamin HCl (vitamin B1), and combinations thereof.

“Solid,” as used herein, can refer to any solid formulation of the composition of the present invention. For example, the compositions of the present invention can be formulated so as to be in the nature of a solid soap, e.g., in bar form. However, as a person of skill in the art would appreciate, any solid formulation that can be employed consistent with the effectiveness of the compositions can be used in accordance with the present invention.

“Liquid,” as used herein, can refer to any liquid formulation of the compositions of the present invention. For example, the compositions of the present invention can be formulated so as to be in the nature of a lotion or a body serum. In some embodiments, the liquids will exhibit a foaming action when applied to the skin and left for a period of time.

“Administer” refers to causing a composition of the present invention to be delivered to a patient in such a manner that the composition can be therapeutically effective for its intended purpose. For example, “administer” includes, without limitation, the application of the compositions to the skin of the patient, i.e., topically.

Generally, the names used to refer to various active ingredients should be understood to refer to all derivatives, salts, metabolites, and other variants that have similar biological activity to the ingredient named. In addition, where appropriate, the names are also intended to refer to extracts thereof. Unless specified or indicated by the context that other units are appropriate, all concentrations expressed as percentages are by weight, which is also expressed herein as “w/w.”

The compositions of the present invention can be administered to any mammal in need of the composition that can experience the beneficial effects of the compositions of the invention. Any such mammal is considered a “patient.” Such patients include humans and non-humans, such as pets and farm animals. “Patient” includes those that are receiving the medication in the absence of medical supervision, i.e., those that acquire and/or use the compositions without a prescription. The term “patient” includes humans of all ages. For example “patient” includes adolescents, i.e., those from about 11 years to about 22 years of age. However, as a person of skill in the art would appreciate, humans other than adolescents suffer from acne, and are properly referred to as patients. In some embodiments the present invention is directed to methods of treating acne in a patient in need thereof comprising administering a composition of the present invention to the patient.

### Compositions

In one aspect, the present invention provides a composition comprising a topical vehicle, nanosilver, acne medicine, and soothing agents, effective for treatment of acne without irritation.

Silver compounds have been used in the treatment or prevention of various diseases. For instance, silver nitrate has recognized therapeutic uses. Physicians have used silver nitrate successfully in the treatment of skin ulcers, compound fractures and suppurating (draining puss) wounds, and dilute silver nitrate is applied in the eye of neonates to prevent gonorrheal ophthalmia. Silver foil dressings have been used to dress wounds, and silver-coated fabrics have been used for the treatment of complex bone infections. Without wishing to be bound by any particular theory, it is believed that silver exerts its antibacterial and antimicrobial effects because a small amount of silver is released from the metallic surface when placed in contact with liquids. As used herein, an “antibacterial” composition is effective against bacteria. As used herein, an “antimicrobial” composition is effective against bacteria, viruses and/or fungi.

Silver is capable of destroying bacteria, viruses, and fungi by inhibiting the organism's metabolism of oxygen. This action essentially suffocates the pathogen. The cells in many multicellular organisms, including those of humans, are largely unaffected by treatment. Upon exposure to silver, the invading cell is suffocated, and is cleared out of the body by the immune, lymphatic and elimination systems. Nanosilver is effective against the nitrogen breathing bacteria which are one of the causes of acne.

In order to more fully understand how silver nanoparticles kill pathogens, an understanding of how bacteria, viruses and fungi live and grow is required.

Bacteria: All bacteria use an enzyme as a form of “chemical lung” in order to metabolize oxygen. Silver ions cripple the enzyme and stop the uptake of oxygen. This effectively suffocates any bacteria, rapidly killing them (for example, in about 6 minutes) and leaving surrounding tissue or material largely unaffected.

Viruses: Viruses grow by taking over another living cell and reprogramming the nucleus to replicate the virus. Nanosilver can be effective against virus-infected cells.

Fungus: A fungus is composed of a series of single cells. Each cell survives with the help of a “chemical lung” similar to that seen in bacteria. As in bacteria, the presence of silver ions disables the chemical lung and the fungus dies.

Drug Resistant Pathogens: Silver nanoparticles have an advantage over antibiotics and many other antibacterial and antimicrobial substances in that pathogens can develop resistance to many drug therapies, but research to date has failed to find any evidence of resistance to silver such as that used in the compositions and methods of the present invention. Silver nanoparticles kill all types of fungal infections, bacteria and viruses, including those involving antibiotic-resistant strains. In addition, whereas no drug-based antibiotic is effective on all types of bacteria, silver nanoparticles are broadly effective.

In addition to killing pathogens, silver also stimulates proper wound healing. It may also aid in repairing some nerve damage.

Manufacturing entire objects from pure silver metal or coating them with silver is prohibitively expensive for consumer items, but impregnating other materials with silver nanoparticles is a practical way to exploit the germ fighting properties of silver. Although silver is known to be effective in very low concentrations, a smaller particle size is more effective because of its large surface to volume ratio. Silver can only be as effective as the amount of its surface area that comes into contact with the pathogen. Nano-scale silver particles, for example those in the 5-100 nanometer size range, can possess far more effective antibacterial and antimicrobial action because of its increased surface-to-volume ratio compared to larger-sized particles.

The extremely small size of silver nanoparticles means they exhibit different as well as unexpectedly superior properties when compared with the bulk material. Their large surface area relative to their volume allows them to easily interact with other particles and increases their antibacterial efficiency. This effect can be so great that one gram of silver nanoparticles can be effective in conferring antibacterial properties to hundreds of square meters of substrate material. It has been discovered that compositions of the present invention comprising silver nanoparticles are surprisingly effective in treating acne when compared to previously available acne medication products. In addition, nano-sized particles are also more easily dispersed into various solutions, enabling them be effective in skin systems such as those of the human body. As a result, nanosilver, whether or not delivered as part of a delivery system, is highly active in acne treatment.

In some embodiments, the nanosilver concentration ranges from about 0.5 to about 500 parts per million (ppm). In some embodiments, the nanosilver concentration ranges from about 2.5 to about 200 ppm. For example, the nanosilver concentration can be up to or about 0.5, about 1.0, about 1.5, about 2.0, about 2.5, about 3.0, about 3.5, about 4.0, about 4.5, about 5.0, about 5.5, about 6, about 6.5, about 7, about 7.5, about 8, about 8.5, about 9, about 9.5, about 10, about 20, about 30, about 40, about 45, about 50, about 55, about 60, about 70, about 80, about 85, about 90, about 100, about 110, about 120, about 130, about 140, about 150, about 175, about 200 ppm, about 300, about 400, about 500 or greater. As used herein, “about” may refer to a range from 10% below the referenced number to 10% above the referenced number. For example, “about 50” may mean from 45 to 55. Other meanings of “about” may be apparent from the context.

In some embodiments, the nanosilver is incorporated into a delivery system. In some embodiments, the delivery system concentration ranges from about 0.1% to about 20% (w/w). In some embodiments, the delivery system concentration ranges from about 1% to about 10% (w/w). For example, the nanosilver delivery system can be about 0.1%, about 0.5%, about 1.0%, about 1.5%, about 2.0%, about 2.25%, about 2.5%, about 2.75%, about 3.0%, about 3.5%, about 4.0%, about 4.5%, about 4.75%, about 5%, about 5.25%, about 5.5%, about 6%, about 7%, about 8%, about 9%, about 10% (w/w) or greater.

In some embodiments, the compositions of the present invention comprise salicylic acid. Salicylic acid helps slow down the shedding of cells into follicles, preventing clogging of pores and avoiding or reducing the development of acne. Salicylic acid also helps eliminate existing acne. It works by softening keratin, a protein that forms part of the skin structure. This action helps to loosen dry scaly skin making it easier to remove. Salicylic acid can help to remove the upper layer of skin, allowing other components, such as acne medications, to penetrate more effectively.

In some embodiments, the compositions of the present invention comprise willow bark extract. Willow bark extract contains naturally-produced salicylic acid and exhibits astringent, antiseptic and anti-inflammatory properties. When incorporated into a composition to be applied topically, willow bark extract can eliminate bacteria from the skin, including acne-causing bacteria, and can do so naturally and gently. Willow bark also can act as an astringent, gently toning skin during the cleansing process. In addition to its astringent, antiseptic and anti-inflammatory properties, willow bark also exhibits antipyretic properties and also stimulates circulation in the skin, which can reduce or counteract some of the effects of aging on the skin. An example of a willow bark extract is NAB® Willowbark Extract, available from Arch Personal Care Products, South Plainfield, N.J. Willow bark extract can contain salicylic acid or its precursors in a concentration ranging from about 1% to about 40%, for example about 10% (w/w). In other words, for an exemplary willow bark extract comprising 10% salicylic acid or its precursors, a 5% concentration of willow bark extract delivers 0.5% salicylic acid or its precursors.

In some embodiments, the acne medication can include salicylic acid, benzoyl peroxide, retinoic acid, and/or combinations thereof.

The compositions of the present invention can comprise salicylic acid in an amount from about 0.005% to about 10% (w/w). For example, the compositions can comprise about 0.005%, about 0.01%, about 0.05%, about 0.1%, about 0.2%, about 0.3%, about 0.4%, about 0.45%, about 0.5%, about 0.55%, about 0.6%, about 0.7%, about 0.8%, about 0.9%, about 1%, about 1.1%, about 1.2%, about 1.3%, about 1.4%, about 1.5%, about 1.75%, about 2%, about 2.5%, about 3%, about 4%, about 5%, about 6%, about 7%, about 8%, about 9%, about 10% (w/w) or more salicylic acid. Benzoyl peroxide and retinoic acid can be present in similar amounts. These concentrations apply also to salicylic acid or its precursors contained in willow bark extract. In some embodiments, the salicylic acid concentration ranges from 0.1-5% (w/w), the benzoyl peroxide concentration ranges from 1-10% (w/w), and the retinoic acid concentration ranges from 0.25-4.0 mg or 0.25-4.0% (w/w).

In some embodiments, the soothing agents can include, but are not limited to, sodium palmate, sodium palm kernelate, butyrospermum parkii (i.e., shea butter), menthe piperita (i.e., peppermint) leaf oil, sericin, pyridoxine (a form of vitamin B6), retinyl palmitate and/or other forms of vitamin A), tocopheryl acetate and/or other forms of vitamin E, lauryl laurate, hyaluronic acid, aloe barbadensis leaf juice powder, euterpe oleracea (i.e., acai berry) fruit extract, riboflavin (i.e., vitamin B2), thiamin HCl and/or other forms of vitamin B1, and/or combinations thereof.

The soothing agents can be present in the following amounts: from about 40 to about 70%, for example about 56% (w/w), sodium palmate; about 10% to about 20%, for example about 14% (w/w), sodium palm kernelate; from about 1% to about 10%, for example about 3.5% (w/w), butyrospermum parkii (rhea butter); about 0.1% to about 5%, for example about 1% (w/w), menthe piperita (peppermint) leaf oil; about 0.1% to about 2%, for example about 0.25% (w/w), sericin; from about 0.01% to about 1%, for example about 0.1% (w/w), pyridoxine (vitamin B6); from about 0.01% to about 1%, for example about 0.1% (w/w), retinyl palmitate (vitamin A); from about 0.01% to about 1%, for example about 0.1% (w/w), tocopheryl acetate (vitamin E); from about 0.01% to about 1%, for example about 0.07% (w/w), lauryl laurate; about 0.005% to about 0.5%, for example about 0.045% (w/w), hyaluronic acid; about 0.001% to about 0.1%, for example about 0.02% (w/w), aloe barbadensis leaf juice powder; from about 0.001% to about 0.01%, for example about 0.0025% (w/w), euterpe oleracea (acai berry) fruit extract; from about 0.0001% to about 0.01%, for example about 0.001% (w/w), riboflavin (vitamin B2); and/or about 0.0001% to about 0.01%, for example about 0.001% (w/w), thiamin HCl (vitamin B1).

In some embodiments, the vehicle can include, without limitation, water, glycerin, titanium dioxide, zea mays (i.e., corn) starch, hydrolyzed corn starch, hydrozlyzed starch octenylsuccinate, synthetic wax, sodium chloride, cocamidopropyl dimethylamine, hydroxyethyl behenamidopropyl dimonium chloride, pentaerythrityl tetraisostearate, glycerol stearate, tetrasodium EDTA, silica, silica dimethyl silylate, butylene glycol, sodium chondroitin sulfate, atelocollagen, Polyquaternium-67, and/or one or more preservatives (e.g., butylparaben, methylparaben, phenoxyethanol, propylparaben, ethylparaben, and/or isobutylparaben).

The vehicle components can be present in the following amounts: from about 10 to about 20%, for example about 12%, water; from about 1 to about 10%, for example about 6% (w/w), glycerin; from about 0.1 to about 5%, for example about 0.9% (w/w), titanium dioxide; from about 0.1 to about 5%, for example about 0.9% (w/w), zea mays (i.e., corn) starch; from about 0.1 to about 5%, for example about 0.8% (w/w), hydrolyzed corn starch; from about 0.1 to about 5%, for example about 0.8% (w/w), hydrozlyzed starch octenylsuccinate; from about 0.1 to about 5%, for example about 0.7% (w/w), synthetic wax; from about 0.05 to about 2%, for example about 0.3% (w/w), sodium chloride; from about 0.05 to about 2%, for example about 0.3% (w/w), cocamidopropyl dimethylamine; from about 0.05 to about 2%, for example about 0.3% (w/w), hydroxyethyl behenamidopropyl dimonium chloride; from about 0.05 to about 2%, for example about 0.2% (w/w), pentaerythrityl tetraisostearate; from about 0.05 to about 2%, for example about 0.2% (w/w), glycerol stearate; from about 0.001 to about 0.1%, for example about 0.06% (w/w), tetrasodium EDTA; from about 0.0005 to about 0.1%, for example about 0.009% (w/w), silica; from about 0.0005 to about 0.01%, for example about 0.006% (w/w), silica dimethyl silylate; from about 0.0005 to about 0.01%, for example about 0.0025% (w/w), butylene glycol; from about 0.0005 to about 0.01%, for example about 0.002% (w/w), sodium chondroitin sulfate; from about 0.00005 to about 0.01%, for example about 0.0007% (w/w), atelocollagen; from about 0.005% to about 0.2%, for example about 0.07% (w/w), Polyquaternium-67; and/or from about 0.0005 to about 0.1%, for example about 0.0025% (w/w), preservatives.

Other components of the compositions can include, without limitation, citrus medica limonum peel oil, helianthus annuus seed oil, glycine soja oil, iron oxide, camellia sinensis (i.e., white tea) leaf extract, cucumis sativus fruit extract, punica granatum extract, potassium cocoate, potassium olivate, polyhydroxystearic acid, isononyl isononanoate, ethylhexyl isononanoate, sodium cocamidopropyl PG-dimonium chloride phosphate, methyl perfluorobutyl ether, methyl perfluoroisubutyl ether, an acrylates copolymer, disodium PEG-12 dimethicone sulfosuccinate, sodium laroyl sarcosinate, coco-glucoside coconut alcohol, cocamidopropyl betaine, glyceryl cocoate, triethanolamine, phenoxyethanol, ethylhexylglycerin, propylene glycol, camellia sinensis leaf extract, garcinia, mangostana peel extract, punica granatum extract, panthenol, phospholipids, retinyl palmitate, tocopheryl acetate, ascorbyl palmitate, citric acid, hydrolyzed collagen, benzyl PCA, phenoxyethanol, lactic acid, and/or cymbopogon schoenanthus oil.

These other components can be present in the following amounts: from about 0.1% to about 5%, for example about 1.25% (w/w), citrus medica limonum (i.e., lemon) peel oil; from about 0.1% to about 5%, for example about 1.1% (w/w), helianthus annuus (i.e., sunflower) seed oil; from about 0.01% to about 0.5%, for example about 0.1% (w/w), glycine soja (i.e., soybean) oil; from about 0.0001% to about 0.01%, for example about 0.02% (w/w), iron oxide; from about 0.0001% to about 0.01%, for example about 0.001% (w/w), camellia sinensis (i.e., white tea) leaf extract; from about 0.0001% to about 0.01%, for example about 0.001% (w/w), cucumis sativus (i.e., cucumber) fruit extract; from about 0.0001% to about 0.01%, for example about 0.001% (w/w), punica granatum extract; from about 5% to about 40%, for example about 20% (w/w), sodium laureth sulfate; from about 1% to about 30%, for example about 10% (w/w), of a mixture comprising potassium cocoate and potassium olivate; from about 1% to about 30%, for example about 10% (w/w), of a mixture comprising polyhydroxystearic acid, isononyl isononanoate, ethylhexyl isononanoate, sodium cocamidopropyl PG-dimonium chloride phosphate, methyl perfluorobutyl ether and methyl perfluoroisubutyl ether; from about 1% to about 30%, for example about 9% (w/w), of carbopol Aqua SF1 (an acrylates copolymer); from about 0.5% to about 20%, for example about 5% (w/w), disodium PEG-12 dimethicone sulfosuccinate; from about 0.5% to about 20%, for example about 5% (w/w), sodium laroyl sarcosinate; from about 0.1% to about 10%, for example about 3% (w/w), of a mixture comprising coco-glucoside and coconut alcohol; from about 0.1% to about 10%, for example about 3% (w/w), cocamidopropyl betaine; from about 0.1% to about 10%, for example about 3% (w/w), PEG-7 glyceryl cocoate; from about 0.1% to about 10%, for example about 1.25% (w/w), triethanolamine; from about 0.1% to about 10%, for example about 1% (w/w), of a mixture comprising phenoxyethanol and ethylhexylglycerin; from about 0.01% to about 5%, for example about 0.5% (w/w), of a mixture comprising propylene glycol, camellia sinensis leaf extract, garcinia, mangostana peel extract and punica granatum extract; from about 0.01% to about 5%, for example about 0.5% (w/w), panthenol; from about 0.01% to about 5%, for example about 0.5% (w/w), of a mixture comprising water, phospholipids, retinyl palmitate, tocopheryl acetate and ascorbyl palmitate; citric acid; from about 0.1% to about 10%, for example about 2% (w/w), hydrolyzed collagen; from about 0.1% to about 10%, for example about 1%, of a mixture comprising benzyl PCA and phenoxyethanol; from about 0.1% to about 10%, for example about 1% (w/w), lactic acid; from about 0.01% to about 5%, for example about 0.125% (w/w), cymbopogon schoenanthus (i.e., lemongrass) oil; and/or combinations thereof.

The compositions of the invention can be in solid or in liquid form. In some embodiments, the composition can include moisturizing agents. In some embodiments, it can include cleansing agents. “Moisturizing agent” refers to any ingredient that preserves or increases the moisture content of the skin. Examples of moisturizing agents include, without limitation, hyaluronic acid, aloe, sericin, chitosan, and/or collagen. “Cleansing agent” refers to any ingredient that removes dirt, dead cells, or any other substance that can clog pores, and is typically a surfactant. Examples of cleansing agents include, without limitation, sodium palmate, sodium palm kernelate, and/or sodium laureth sulfate.

In some embodiments, the nanosilver can be delivered in a nanosilver delivery system. This composition can be in liquid form, for example in the form of a liquid cleanser. The liquid cleanser can be packaged in, for example, pump dispensers. It can also be in solid form, for example in the form of a soap. In some embodiments, the present invention provides a composition comprising nanosilver, collagen, soothing agents and other beneficial ingredients such as willow bark extracts. The composition can be used by men, women and adolescents to kill acne bacteria, reduce break-outs, remove make up, reduce pore size, and exfoliate and tighten skin.

In some embodiments, the present invention may comprise, but is not limited to, the following ingredients: nanosilver in a delivery system, such as nanosilver plated onto silica microspheres and coated with titanium dioxide; vegetable and synthetic base soaps; marine collagen; willow bark extract; chitosan; aloe vera gel (i.e., aloe barbadensis miller); aloe vera juice; vitamins A, B1, B2, B6, B12, C and E; royal jelly; blueberry extract; lemon oil; açai berry; yerba mate; organic yogurt; crushed strawberries; peppermint oil; cucumber juice; pomegranate; goji berry; white tea; green tea; red tea; grape seed extract; sericin; and triclosan.

In some embodiments, the present invention provides a composition comprising vitamin-enhanced antioxidant ingredients in an aloe juice base instead of distilled water, for use as a therapeutic skin moisturizer for dry skin. In some embodiments, the composition can comprise blueberry extracts, collagen, and virgin coconut oil. In some embodiments, the composition can be formulated as a therapeutic skin treatment for oily skin. In some embodiments, the composition can comprise blueberry extracts, collagen, organic yogurt, crushed strawberries, lime and cucumber juice.

In some embodiments, the compositions of the present invention comprise hyaluronic acid (HA). HA can be replaced in any embodiment of the invention with salts or other derivatives thereof, such as sodium hyaluronate. HA helps retain the skin's natural moisture, tightens the skin and reduces pore size. HA can be found naturally in most every cell in the body, including in skin tissue. It is found in both the deep underlying dermal areas as well as the visible epidermal top layers. Young skin is smooth and elastic and contains large amounts of HA, which helps keep the skin young and healthy in appearance. Because HA can bind up to about 1000 times its weight in water, it can serve as a source of moisture for the skin. With age, the ability of the skin to produce HA decreases, which can lead to the wrinkled appearance of skin in older patients as well as other effects.

In some embodiments, the compositions of the present invention comprise aloe vera, also referred to herein as “aloe.” Both “aloe vera” and “aloe” are used herein to refer to all forms and extracts of aloe vera, including aloe barbadensis leaf juice powder, as well as biologically active variants thereof. Aloe vera contains about 75 nutrients, 200 active compounds, 20 essential minerals, 18 amino acids, and 12 vitamins. It can serve as a hydrator and soothing agent, which can provide beneficial effects to damaged, dry, chapped or sun-exposed skin. Aloe can reduce or counteract the effects of the aging process on skin in several ways. For example, aloe removes dead skin cells, wastes, and toxins from the skin, including from the pores. Aloe stimulates proteolytic enzyme action in skin tissue, enhancing the process of cell division. Specifically, fibroblast cells, which manufacture the collagen needed to keep the skin firm, are produced six to eight times faster in cells treated with aloe than in cells not so treated. The vitamins, minerals, and amino acids in aloe provide nourishment to skin cells and also help to moisturize it. Aloe acts as a moisturizing agent by slowing evaporation from the skin and drawing moisture from the air into the skin. Aloe penetrates the outer skin surface, which allows it to exert its effects deep in skin tissue.

In some embodiments, the compositions of the present invention comprise sericin. Sericin can be derived from silk worms. Sericin binds with the skin's keratin as well as with other proteins, forming a protective layer. Sericin also binds with water to help regulate the moisture balance of the skin. It diminishes the appearance of facial lines and helps skin feel soft and smooth to the touch.

In some embodiments, the compositions of the present invention comprise chitosan. Chitosan is a deacetylated breakdown product of chitin, which is an abundant natural polysaccharide polymer. Chitosan has the properties of a gel, which permits it to bind to water and increase the moisture content of the skin, which can help to treat and prevent skin dryness. Chitosan also has an anti-microbial effect.

In some embodiments, the compositions of the present invention comprise acai berries and/or variants and extracts thereof. As used herein, “acai berry” is used to refer to the berry itself as well as all forms, derivatives and extracts thereof. Açai berries come from the acai palm, also known as Euterpe oleracea, and provide antioxidants, which help to reduce or prevent oxidative damage to skin tissues, which contributes to the appearance of aging. The berry extract can comprise flavonoids, which can fight inflammation. Acai berry also contains essential amino and fatty acids, which can contribute to skin regeneration, and phytosterols, which can contribute to the preservation of skin collagen.

In some embodiments, the compositions of the present invention comprise white and green tea extracts. As used herein, “white tea” and “green tea” are also used to refer to the extracts thereof. White and green tea extracts exhibit potent anti-oxidant properties. Many of the beneficial properties of these extracts result from the presence of flavonoid photochemicals called polyphenols, in particular the group of polyphenols known as catechins. Such catechins include gallocatechin (GC), epigallocatechin (EGC), epicatechin (EC), and epigallocatechin gallate (EGCG). White and green tea extracts can also exhibit anti-inflammatory activities. Application of 2% polyphenone (found in, for example, some green tea extracts) is effective against papulopustular rosacea, which is characterized by redness, inflammation, and papule and pustule formation on facial skin. Polyphenone contributes to a significant reduction in inflammatory lesions. Topical green tea polyphenols can be effective at slowing down the development of some signs of aging. White and green tea extracts can also include ellagic acid.

In some embodiments, the compositions of the present invention comprise vitamin A. As used herein, “vitamin A” refers also to chemically active variants thereof, including, without limitation, retinyl palmitate. Vitamin A strengthens the protective tissue of the skin and can prevent acne. It also plays a role in the maintenance and repair of skin and mucous tissue. It can also help reduce sebum production. Vitamin A also exhibits antioxidant properties. A deficiency in vitamin A is associated with increased prevalence of acne. Decreased levels of vitamin A in tissues can lead to the oxidation of unsaturated fatty acids in the cell, which can cause toxic effects in the cell.

In some embodiments, the compositions of the present invention comprise vitamin C. Vitamin C, also known as L-ascorbic acid, is required for numerous metabolic functions in the body, including many associated with tissue growth and repair. Vitamin C can help protect against infection and enhance the functioning of the immune system. Vitamin C can also boost collagen synthesis by fibroblasts.

In some embodiments, the compositions of the present invention comprise vitamin E. As used herein, “vitamin E” refers also to chemically active variants thereof, including, without limitation, tocopheryl acetate. Vitamin E plays a role in the protection of cell membranes from damage. It exhibits antioxidant properties helping to maintain healthy skin by reducing or eliminating free radicals generated during cellular metabolism. Vitamin E can help skin recover from acne scarring and reduce the appearance of scars left by acne. Vitamin E also plays a role in the regulation of vitamin A levels in humans. Acne in both men and women improves with vitamin E treatment.

In some embodiments, the compositions of the present invention comprise marine collagen. Collagen is the main supporting fiber in the dermis layer of the skin, providing strength and structure. Topical collagen can act as a moisturizing agent. The compositions of the present invention can also comprise cucumber, which promotes a healthy glow in skin. The compositions can also comprise antioxidants, which can help prevent or reduce damage to skin caused by free radials. Free radicals cause damage by contributing to oxidative degradation. Oxidation causes many of the visible signs of aging in the skin. The compositions can also comprise essential oils, which have antiseptic and anti-inflammatory properties. The compositions can also comprise one or more of vitamin B1, B2, and B6. The B vitamins play a role in cellular metabolism, which can enhance the immune system and encourage cell growth and division. The compositions can also comprise pomegranate. Pomegranate exhibits anti-oxidant properties. It may also be effective against certain forms of cancer. Vitamin B1 can be in the form of, for example, thiamin HCl, though a person of skill in the art would appreciate that other forms of vitamin B1 are included within the scope of the invention. Vitamin B2 is also referred to herein as “riboflavin,” and either “vitamin B2” or “riboflavin” is intended to refer also to biologically active variants thereof. Vitamin B6 can be in the form of, for example, pyridoxine, though a person of skill in the art would appreciate that other forms of this and the other B vitamins are included within the scope of the invention.

### Methods of Treating

In another aspect, the present invention provides methods of treating acne in a patient in need thereof comprising administering a therapeutically effective amount of one of the compositions of the present invention to the patient, thereby treating the acne. The compositions can be administered topically.

In some embodiments, the composition is applied to the patient's face and/or body and remains there for at least about six minutes. In some embodiments, the effectiveness of the nanosilver is maximized if the compositions are left on the patient's face and/or body for about this length of time. However, the invention is not limited to this time frame. The compositions can be applied for longer or shorter periods of time, for example about 1, about 2, about 3, about 4, about 5, about 6, about 7, about 8, about 9, about 10 minutes or longer, provided the time frame is consistent with the composition's effectiveness. In some embodiments, the compositions exhibit a foaming effect after several minutes, which can serve as an indicator to the patient that some ingredients in the composition are working efficaciously.

In some embodiments, the methods of the present invention comprise applying one of the compositions of the present invention at least about 3-4 times daily to the face and/or body of the patient until the severity of the patient's acne is significantly reduced, after which time the composition is applied at least twice daily. In some embodiments, the methods of the present invention comprise applying one of the compositions of the present invention 1, 2, 3 or 4 or more times per week, or 1, 2, 3, or 4 times per day, as necessitated by the severity of the patient's acne. Treatment according to the compositions and methods of the present invention can continue for one day or more, one week or more, one month or more, one year or more, or indefinitely.

In some embodiments, the patient is a human of adolescent age. However, humans of other ages suffer from acne or experience one or more symptoms of acne. Treatment of any such person with the disclosed compositions and methods is within the scope of the invention.

### Kits

In some embodiments, the present invention provides a kit comprising a composition of the present invention in a dispenser. The dispenser can be any suitable container or other containing means that is capable of holding and dispensing the composition and which will not significantly interact with the composition. The dispenser can be, for example, a plastic bottle, a vial, a glass container, a squeezable container such as one with a resealable enclosure, a foil packet, or other suitable containers, as would be appreciated by a person of skill in the art.

In some embodiments, the kit further comprises one or more printed labels. The printed label is functionally related to the composition/dispenser in that it may provide instructions for administering any of the compositions, using any of the kits, or performing any other method herein described. The labeling instructions will be consistent with the methods of treatment described herein. The labeling may be associated with the dispenser by any means that maintain a physical proximity of the two, by way of non-limiting example, they may both be contained in a packaging material such as a box or plastic shrink wrap or may be associated with the instructions being bonded to the dispenser such as with glue that does not obscure the labeling instructions or other bonding or holding means. Such labeling can provide instructions for, for example, applying the composition in the dispenser topically to a patient's face and/or body and leaving it on the face and/or body for at least about six minutes.

Notably, it has previously been contraindicated to prepare and administer nanosilver and acne medications such as salicylic acid together in one composition, as nanosilver requires a five- to six-minute ‘incubation’ time on the skin, while in contrast acne medications such as salicylic acid are to be immediately removed to avoid excessive dryness and irritation, which can lead to further breakouts. Surprisingly, the use of the inventive combinations with soothing ingredients permit the simultaneous use of nanosilver and acne medication such as salicylic acid in the compositions and methods of the present invention. Accordingly, acne medications such as salicylic acid can be used in the recommended full-strength dose and the slower-acting nanosilver can be kept on the skin for the proper amount of time due to the action of the soothing components of this invention. Furthermore, the foaming action of some embodiments can provide increased coverage when compared with non-foaming reagents and the time delay by which this foaming is achieved can be used to indicate to the patient when the compositions can be removed from the skin.

According to some embodiments of the present invention, the combination of nanosilver with acne medications such as salicylic acid and other ingredients provide surprising effectiveness and exhibit unexpectedly superior results in the treatment, reduction and avoidance of acne, acne breakouts and related symptoms and conditions. Without wishing to be bound by any particular theory, the nanosilver is effective at killing acne-causing bacteria, and the salicylic acid and other ingredients exhibit drying and exfoliating effects. The compositions of the present invention comprising nanosilver, salicylic acid and other ingredients exhibit surprising efficacy, such as would not have been predicted prior to the present invention.

Soothing agents included in some embodiments of the compositions of the present invention calm and heal the skin during the acne eruptions. Previously available acne products are made of numerous chemicals which can have deleterious effects on skin, for example drying out the skin or leaving it with a red and unbalanced color. The inventive combinations of ingredients disclosed herein exhibit surprisingly superior results in promoting the healing process as well as reducing pore size and tightening the skin.

The following examples are further illustrative of the present invention, but are not to be construed to limit the scope of the present invention.

### EXAMPLES

### Example 1

### Acne Treatment Compositions

Example 1A: Solid acne treatment composition. An example solid acne treatment composition for topical application is presented in Table 1A below. The composition treats acne when an effective amount is applied to the skin according to the methods of the present invention.

TABLE 1A
Solid acne treatment composition.

| Ingredient | Percentage (w/w) |
|---|---|
| Sodium Palmate | 56.2158 |
| Sodium Palm K ernelate | 14.0540 |
| Water | 12.4637 |
| Glycerin | 5.7984 |
| Butyrospermum Parkii (Shea Butter) | 3.5450 |
| Mentha Piperita (Peppermint) Leaf Oil | 1.0000 |
| Titanium Dioxide | 0.9005 |
| Zea Mays (Corn) Starch | 0.8787 |
| Hydrolyzed Corn Starch | 0.8121 |
| Hydrolyzed Starch Octenylsuccinate | 0.7787 |
| Synthetic Wax | 0.6950 |
| Salicylic Acid | 0.5010 |
| Sodium Chloride | 0.3534 |
| Cocamidopropyl Dimethylamine | 0.3507 |
| Hydroxyethyl Behenamidopropyl Dimonium Chloride | 0.3450 |
| Sericin | 0.2500 |
| Pentaerythrityl Tetraisostearate | 0.2363 |
| Glyceryl Stearate | 0.2171 |
| Pyridoxine | 0.1000 |
| Retinyl Palmitate | 0.1000 |
| Tocopheryl Acetate | 0.1000 |
| Polyquaternium-67 | 0.0867 |
| Lauryl Laurate | 0.0700 |
| Tetrasodium EDTA | 0.0552 |
| Hyaluronic Acid | 0.0450 |
| Aloe Barbadensis Leaf Juice Powder | 0.0200 |
| Silica | 0.0090 |
| Silica Dimethyl Silylate | 0.0063 |
| Butylene Glycol | 0.0025 |
| Euterpe Oleracea (Acai Berry) Fruit Extract | 0.0025 |
| Sodium Chondroitin Sulfate | 0.0018 |
| Riboflavin | 0.0010 |
| Thiamin HCl | 0.0010 |
| Atelocollagen | 0.0007 |
| Nanosilver | 0.0005 |
| Preservatives | 0.0024 |

Example 1B: Solid acne treatment composition. Another example solid composition for topical acne treatment is presented in Table 1B below. The composition treats acne when an effective amount is applied to the skin according to the methods of the present invention.

TABLE 1B
Solid acne treatment composition.

| Ingredient | Concentration |
|---|---|
| Nanosilver | 100 ppm |
| Natural base, palm oil, palm kernel oil | Up to 100% |
| Salicylic acid | 0.50% |
| Sericin | 0.25% |
| Marine collagen | 0.25% |
| Aloe vera | 2% |
| Shea buter | 1.50% |
| Acai berries | 0.25% |
| Hyaluronic acid | 3.00% |
| Vitamins A, B2, B6, E | 0.1% each |
| Peppermint essential oil | |

Example 1C: Liquid acne treatment composition. An example liquid acne treatment composition for topical application is presented in Table 1C below. The composition treats acne when an effective amount is applied to the skin according to the methods of the present invention.

TABLE 1C
Liquid acne treatment composition.

| Description | % |
|---|---|
| Water | Q.S. to 100% |
| Sodium Laureth Sulfate | 20.000 |
| Glycerin | 10.000 |
| Potassium Cocoate and Potassium Olivate | 10.000 |
| Mixture comprising Polyhydroxystearic Acid, Isononyl | 10.000 |
| Isononanoate, Ethylhexyl Isononanoate, Sodium | |
| Cocamidopropyl PG-Dimonium Chloride Phosphate, | |
| Methyl Perfluorobutyl Ether and Methyl | |
| Perfluoroisobutyl Ether | |
| Acrylates Copolymer | 9.000 |
| Disodium PEG-12 Dimethicone Sulfosuccinate | 5.000 |
| Sodium Lauroyl Sarcosinate | 5.000 |
| Silver, Silica and Titanium Dioxide | 5.000 |
| Coco-Glucoside and Coconut Alcohol | 3.000 |
| Cocamidopropyl Betaine | 3.000 |
| PEG-7 Glyceryl Cocoate | 3.000 |
| Sodium Hyaluronate | 3.000 |
| Triethanolamine | 1.250 |
| Salicylic Acid | 1.000 |
| Phenoxyethanol and Ethylhexylglycerin | 1.000 |
| Sericin | 0.500 |
| Mixture comprising Propylene Glycol, Camellia Sinensis | 0.500 |
| Leaf Extract and Garcinia | |
| Mangostana Peel Extract and Punica Granatum Extract | |
| Panthenol | 0.500 |
| Mixture comprising Water, Phospholipids, Retinyl | 0.500 |
| Palmitate, Tocopheryl Acetate, and Ascorbyl Palmitate | |
| Mentha Piperita (Peppermint) Oil | 0.200 |
| Aloe Barbadensis Leaf Juice | 0.125 |
| Niacinamide | 0.100 |
| Red 4 | 0.045 |
| Yellow 5 | 0.034 |
| Citric Acid | Q.S. |

Example 1D: Liquid acne treatment formula. Table 1D presents an example liquid acne treatment composition for topical application. The composition treats acne when an effective amount is applied to the skin according to the methods of the present invention.

TABLE 1D
Liquid acne treatment composition.

| Ingredient | Concentration |
|---|---|
| Nanosilver | 50 ppm |
| Salicylic acid | 1% |
| 90% coconut oil, 10% pomace grade olive oil | |
| Hyaluronic acid | 3% |
| Chitosan | 0.5% |
| Sericin | 0.5% |
| Acai berries | 0.5% |
| Marine collagen | 1% |
| Aloe vera gel | 5% |
| Acai berries | 0.5% |
| White-green tea blend | 0.5% |
| Vitamins B1, B2, B6 blend | |
| Vitamins A, E, C blend | |
| Peppermint essential oil | |

### Example 2

### Skin Treatment Compositions

Example 2A: Solid skin treatment composition. An example solid skin treatment composition for topical application is presented in Table 2A below. The composition treats acne when an effective amount is applied to the skin according to the methods of the present invention.

TABLE 2A
Solid skin treatment composition.

| Ingredient | Percentage % |
|---|---|
| Sodium Palmate | 56.486 |
| Sodium Palm Kernelate | 14.121 |
| Water | 12.561 |
| Glycerin | 5.885 |
| Butyrospermum Parkii (Shea Butter) | 3.045 |
| Citrus Medica Limonum (Lemon) Peel Oil | 1.250 |
| Helianthus Annuus (Sunflower) Seed Oil | 1.089 |
| Pentaerythrityl Tetraisostearate | 0.945 |
| Synthetic Wax | 0.695 |
| Zea Mays (Corn) Starch | 0.695 |
| Hydrolyzed Corn Starch | 0.595 |
| Hydrolyzed Starch Octenylsuccinate | 0.595 |
| Sodium Chloride | 0.355 |
| Hydroxyethyl Behenamidopropyl Dimonium Chloride | 0.345 |
| Sericin | 0.250 |
| Titanium Dioxide | 0.226 |
| Pyridoxine | 0.100 |
| Retinyl Palmitate | 0.100 |
| Thiamin HCl | 0.100 |
| Tocopheryl Acetate | 0.100 |
| Glycine Soja (Soybean) Oil | 0.099 |
| Lauryl Laurate | 0.070 |
| Polyquaternium-67 | 0.070 |
| Tetrasodium EDTA | 0.055 |
| Hyaluronic Acid | 0.045 |
| Silica Dimethyl Silylate | 0.025 |
| Aloe Barbadensis Leaf Juice Powder | 0.020 |
| Orange 4 | 0.020 |
| Butylene Glycol | 0.010 |
| Salix Alba (Willow) Bark Extract | 0.010 |
| Silica | 0.009 |
| Sodium Chondroitin Sulfate | 0.007 |
| Atelocollagen | 0.003 |
| Euterpe Oleracea (Acai Berry) Fruit Extract | 0.003 |
| Iron Oxide | 0.002 |
| Preservatives | 0.012 |
| Silver | 0.001 |
| Camellia Sinensis (White Tea) Leaf Extract | 0.001 |
| Cucumis Sativus (Cucumber) Fruit Extract | 0.001 |
| Punica Granatum Extract | 0.001 |
| Riboflavin | 0.001 |

Example 2B: Solid skin treatment composition. Table 2B presents another example solid skin treatment composition for topical application. The composition treats acne when an effective amount is applied to the skin according to the methods of the present invention.

TABLE 2B
Solid skin treatment composition.

| Ingredient | Concentration |
|---|---|
| Nanosilver | 100 ppm |
| Marine collagen | 1% |
| Aloe vera | 2% |
| Shea butter | 1.50% |
| Acai berries | 0.25% |
| Hyaluronic acid | 3.00% |
| White-green tea blend | 0.10% |
| Cucumber juice | 0.10% |
| Pomegranate | 0.10% |
| Willow bark extract | 1% |
| Vitamins B1, B2 | |
| Vitamin B6 | |
| Vitamins A, E, C blend | |
| Lemon grass essential oil | 0.125% |

Example 2C: Liquid skin treatment composition. Table 2C presents an example liquid skin treatment composition for topical application. The composition treats acne when an effective amount is applied to the skin according to the methods of the present invention.

TABLE 2C
Liquid skin treatment composition.

| Description | % |
|---|---|
| Water | Q.S. to 100% |
| Sodium Laureth Sulfate | 20.0000 |
| Glycerin | 10.0000 |
| Potassium Cocoate and Potassium Olivate | 10.0000 |
| Cocamidopropyl Betaine | 10.0000 |
| Acrylates Copolymer | 9.0000 |
| Disodium PEG-12 Dimethicone Sulfosuccinate | 5.0000 |
| Sodium Lauroyl Sarcosinate | 5.0000 |
| Coco-Glucoside and Coconut Alcohol | 3.0000 |
| Sodium Hyaluronate | 3.0000 |
| Silver, Silica and Titanium Dioxide | 2.5000 |
| Mixture comprising Polyhydroxystearic Acid, Isononyl | 2.5000 |
| Isononanoate, Ethylhexyl Isononanoate, Sodium | |
| Cocamidopropyl PG-Dimonium Chloride Phosphate, | |
| Methyl Perfluorobutyl Ether and Methyl | |
| Perfluoroisobutyl Ether | |
| Hydrolyzed Collagen | 2.0000 |
| Triethanolamine | 1.2500 |
| Water and Salix Nigra (Willow) Bark Extract | 1.0000 |
| Benzyl PCA and Phenoxyethanol | 1.0000 |
| Lactic Acid | 1.0000 |
| Sericin | 0.5000 |
| Cucumis Sativus (Cucumber) Fruit Extract in Glycerin | 0.5000 |
| and Water | |
| Euterpe Oleracea Fruit Extract in Glycerin and Water | 0.5000 |
| Mixture comprising Propylene Glycol, Camellia Sinensis | 0.5000 |
| Leaf Extract, Garcinia Mangostana Peel Extract | |
| and Punica Granatum Extract | |
| Mixture comprising Water, Phospholipids, Retinyl | 0.5000 |
| Palmitate, Tocopheryl Acetate, and Ascorbyl Palmitate | |
| Yellow 5 | 0.4100 |
| Red 4 | 0.2100 |
| Aloe Barbadensis Leaf Juice | 0.1250 |
| Cymbopogon Schoenanthus Oil | 0.1250 |
| Niacinamide | 0.1000 |
| Pyridoxine HCl | 0.0001 |
| Riboflavin | 0.0001 |
| Thiamine HCl | 0.0001 |

Example 2D: Liquid skin treatment composition. Table 2D presents an example liquid skin treatment composition for topical application. The composition treats acne when an effective amount is applied to the skin according to the methods of the present invention.

TABLE 2D
Liquid skin treatment composition.

| Ingredient | Concentration |
|---|---|
| Nanosilver | 50 ppm |
| 90% coconut oil and 10% pomace grade olive oil | |
| Hyaluronic acid | 3% |
| Chitosan | 0.5% |
| Sericin | 0.5% |
| Acai berries | 0.5% |
| Marine collagen | 2% |
| Aloe vera gel | 5% |
| White-green tea blend | 0.5% |
| Cucumber juice | 0.5% |
| Pomegranate | 0.5% |
| Willow bark extract | 1% |
| Vitamins B1, B2, each reduced by 75% | |
| Vitamin B6 | |
| Vitamins A, E, C blend | |
| Lemon grass essential oil | 0.125% |

### Example 3

### Efficacy Study

Several subjects who are suffering from acne are enrolled in a placebo-controlled split-face efficacy study of the compositions of the present invention. One-half of each subject's face is treated with a composition of the present invention. The other half of the subject's face is treated with a placebo. During each treatment, the inventive composition and the placebo are applied to the respective side of the subject's face and are allowed to remain for approximately six minutes. This treatment is repeated 2-3 times daily for several days. The severity of the subjects' acne is significantly reduced on the side that is treated with the inventive composition.

These examples illustrate possible embodiments of the present invention. As one of skill in the art will appreciate, because of the versatility of the compositions and methods of using the compositions disclosed herein, the compositions and methods can be used in other similar ways to those described herein. Thus, while the invention has been particularly shown and described with reference to some embodiments thereof, it will be understood by those skilled in the art that they have been presented by way of example only, and not limitation, and various changes in form and details can be made therein without departing from the spirit and scope of the invention. Therefore, the breadth and scope of the present invention should not be limited by any of the above-described exemplary embodiments, but should be defined only in accordance with the following claims and their equivalents.

All documents cited herein, including journal articles or abstracts, published or corresponding U.S. or foreign patent applications, issued or foreign patents, or any other documents, are each entirely incorporated by reference herein, including all data, tables, figures, and text presented in the cited documents.

1. A composition comprising a topical vehicle, nanosilver, acne medicine, and one or more soothing agents in such amounts that the composition is effective for treatment of acne without irritation.

2-3. (canceled)

4. The composition of claim 1, further comprising one or more moisturizing agents.

5. The composition of claim 1, wherein the composition does not comprise moisturizing agents.

6. The composition of claim 1, further comprising one or more cleansing agents.

7. The composition of claim 1, wherein the composition does not comprise cleansing agents.

8. The composition of claim 1, wherein the nanosilver is delivered in a nanosilver delivery system.

9. The composition of claim 1, wherein the nanosilver concentration ranges from about 50 to about 1000 ppm.

10. The composition of claim 1, wherein the nanosilver concentration is about 100 ppm.

11. The composition of claim 1, wherein the acne medicine is selected from the group consisting of salicylic acid, benzoyl peroxide, retinoic acid, and combinations thereof.

12. The composition of claim 11, wherein the salicylic acid concentration ranges from 0.1-5% (w/w), the benzoyl peroxide concentration ranges from 1-10% (w/w), and/or the retinoic acid content ranges from 0.25-4.0% (w/w).

13. (canceled)

14. The composition of claim 1, wherein the one or more soothing agents comprise one or more of from about 40% to about 70% (w/w) sodium palmate, from about 10% to about 20% (w/w) sodium palm kernelate, from about 1% to about 10% (w/w) butyrospermum parkii (shea butter), from about 0.1% to about 5% (w/w) menthe piperita (peppermint) leaf oil, from about 0.1% to about 2% (w/w) sericin, from about 0.01% to about 1% (w/w) pyridoxine (vitamin B6), from about 0.01% to about 1% (w/w) retinyl palmitate (vitamin A), from about 0.01% to about 1% (w/w) tocopheryl acetate (vitamin E), from about 0.01% to about 1% (w/w) lauryl laurate, from about 0.005% to about 0.5% (w/w) hyaluronic acid, from about 0.001% to about 0.1% (w/w) aloe barbadensis leaf juice powder, from about 0.001% to about 0.01% (w/w) euterpe oleracea (acai berry) fruit extract, from about 0.0001% to about 0.01% (w/w) riboflavin (vitamin B2), and from about 0.0001 to about 0.01% (w/w) thiamin HCl (vitamin B1).

15. (canceled)

16. The composition of claim 1, wherein the vehicle comprises comprising one or more of from about 10% to about 20% (w/w) water, from about 1% to about 10% (w/w) glycerin, from about 0.1% to about 5% (w/w) titanium dioxide, from about 0.1% to about 5% (w/w) zea mays (i.e., corn) starch, from about 0.1% to about 5% (w/w) hydrolyzed corn starch, from about 0.1% to about 5% (w/w) hydrolyzed starch octenylsuccinate, from about 0.1% to about 5% (w/w) synthetic wax, from about 0.05% to about 2% (w/w) sodium chloride, from about 0.05% to about 2% (w/w) cocamidopropyl dimethylamine, from about 0.05% to about 2% (w/w) hydroxyethyl behenamidopropyl dimonium chloride, from about 0.05% to about 2% (w/w) pentaerythrityl tetraisostearate, from about 0.05% to about 2% (w/w) glycerol stearate, from about 0.001% to about 0.1% (w/w) tetrasodium EDTA, from about 0.0005% to about 0.1% (w/w) silica, from about 0.0005% to about 0.01% (w/w) silica dimethyl silylate, from about 0.0005% to about 0.01% (w/w) butylene glycol, from about 0.0005% to about 0.01% (w/w) sodium chondroitin sulfate, from about 0.00005% to about 0.01% (w/w) atelocollagen, from about 0.005% to about 0.2% (w/w) Polyquaternium-67; and from about 0.0005% to about 0.1% (w/w) preservatives.

17. (canceled)

18. The composition of claim 1, comprising one or more of from about 0.1% to about 5% (w/w) citrus medica limonum peel oil; from about 0.1% to about 5% (w/w) helianthus annuus seed oil; from about 0.01% to about 0.5% (w/w) glycine soja oil; from about 0.0001% to about 0.01% (w/w) iron oxide; from about 0.0001% to about 0.01% (w/w) camellia sinensis leaf extract; from about 0.0001% to about 0.01% (w/w) cucumis sativus fruit extract; from about 0.0001% to about 0.01% (w/w) punica granatum extract; from about 5% to about 40% (w/w) sodium laureth sulfate; from about 1% to about 30% (w/w) of a mixture comprising potassium cocoate and potassium olivate; from about 1% to about 30% (w/w) of a mixture comprising polyhydroxystearic acid, isononyl isononanoate, ethylhexyl isononanoate, sodium cocamidopropyl PG-dimonium chloride phosphate, methyl perfluorobutyl ether and methyl perfluoroisubutyl ether; from about 1% to about 30% (w/w) of an acrylates copolymer; from about 0.5% to about 20% (w/w) disodium PEG-12 dimethicone sulfosuccinate; from about 0.5% to about 20% (w/w) sodium laroyl sarcosinate; from about 0.1% to about 10% (w/w) of a mixture comprising coco-glucoside and coconut alcohol; from about 0.1% to about 10% (w/w) cocamidopropyl betaine; from about 0.1% to about 10% (w/w) PEG-7 glyceryl cocoate; from about 0.1% to about 10% (w/w) triethanolamine; from about 0.1% to about 10% (w/w) of a mixture comprising phenoxyethanol and ethylhexylglycerin; from about 0.01% to about 5% (w/w) of a mixture comprising propylene glycol, camellia sinensis leaf extract, garcinia, mangostana peel extract and punica granatum extract; from about 0.01% to about 5% (w/w) panthenol; from about 0.01% to about 5% (w/w) of a mixture comprising water, phospholipids, retinyl palmitate, tocopheryl acetate and ascorbyl palmitate; from about 0.1% to about 10% (w/w) hydrolyzed collagen; from about 0.1% to about 10% (w/w) of a mixture comprising benzyl PCA and phenoxyethanol; from about 0.1% to about 10% (w/w) lactic acid; and from about 0.01% to about 5% (w/w) cymbopogon schoenanthus oil.

19. A method of treating acne in a patient in need thereof comprising administering a therapeutically effective amount of the composition according to claim 1 to the patient, thereby treating the acne.

20. The method of claim 19, wherein the therapeutically effective amount of the composition is administered topically.

21. The method of claim 19, further comprising the step of permitting the therapeutically effective amount to remain on the patient for at least about six minutes.

22. The method of claim 19, wherein the patient is a human of adolescent age.

23. A composition comprising a topical vehicle, nanosilver, willow bark extract, and one or more soothing agents in such amounts that the composition is effective for treatment of acne without irritation.

24. A kit comprising the composition of claim 1 in a dispenser, wherein a label is associated with the dispenser, and the label indicates that the composition contained in the dispenser is to be used for treating acne.

25. The kit of claim 24, wherein the label provides instructions stating that the composition is to be applied topically to a patient's skin and left on for at least about six minutes.
